(19) United States
(12) Patent Application Publication
(10) Pub. No.: US 2025/0288713 A1
(43) Pub. Date: Sep. 18, 2025

(54) BIORESORBABLE ZINC-BASED WOUND CLOSURE DEVICES

(71) Applicant: THE RESEARCH FOUNDATION FOR THE STATE UNIVERSITY OF NEW YORK, Albany, NY (US)

(72) Inventor: Donghui ZHU, Frisco, TX (US)

(73) Assignee: THE RESEARCH FOUNDATION FOR THE STATE UNIVERSITY OF NEW YORK, Albany, NY (US)

(21) Appl. No.: 18/857,604

(22) PCT Filed: Apr. 20, 2023
(86) PCT No.: PCT/US2023/019209

§ 371 Date: Oct. 17, 2024

Related U.S. Application Data
(60) Provisional application No. 63/333,197, filed on Apr. 21, 2022.

(51) Int. Cl. A61L 17/06; A61B 17/064; A61L 31/02; A61L 31/16; C22C 18/00

(52) U.S. Cl. CPC A61L 17/06; A61B 17/064; A61L 31/022; A61L 31/16; C22C 18/00

(57) ABSTRACT

Bioresorbable zinc-based (i.e., Zn-based) wound closure devices are provided. The Zn-based wound closure devices include Zn and at least one primary alloying element (i.e., Cr, V and/or Zr). The Zn-based wound closure devices are biocompatible with good mechanical strength and tissue compatibility. The Zn-based wound closure devices are safe and easy to use during surgical operations and provide shorter recovery and healing time with less chance of clinical complications. Typical applications for Zn-based wound closure devices include skin wound closure, muscular, vascular, and neuronal anastomosis, as well as GI anastomosis and other tissue or organ anastomosis. The Zn-based wound closure devices can be used for treatment of the above applications for pediatric patients.

### CROSS REFERENCE TO RELATED APPLICATIONS

The present disclosure claims priority to U.S. Provisional Patent Application No. 63/333,197, filed Apr. 21, 2022, which is incorporated herein by reference in its entirety.

### BACKGROUND

The present disclosure relates to wound closure devices, and more particularly to biocompatible and biodegradable surgical wound closure devices for various tissues and organs, and methods for their preparation, as well as their performance of tunable degradation rates to provide closure and better healing.

Each year in America, over 28 million surgeries are performed, and there are about 10 million operations on the digestive system. The anastomotic procedure is one of the key factors determining surgical success. Hand-sewn anastomosis (HA) and stapled anastomosis (SA) comprise the major anastomotic methods in clinical practice of gastrointestinal (GI) surgeries. It's estimated that up to 40% of these operations are SA.

Since the introduction of stapling instruments into surgery, their safety, usability and cost effectiveness have been demonstrated. In colorectal surgery, SA has been shown to be associated with lower complications such as anastomotic leaks, better blood supply, reduced tissue manipulation, less edema, uniformity of sutures and shorter operation time and hospital stay. In upper GI surgery, the use of circular SA in esophagojejunostomy has been demonstrated to be convenient and safe. Compared to conventional HA, SA was also shown to have lower incidence of delayed gastric emptying (DGE) after pancreaticoduodenectomy (PD) without increasing the risk of clinically significant postoperative pancreatic fistula (POPF), anastomotic leak or mortality. Thus, SA is associated with fewer anastomotic complications compared with HA.

In addition, SA can significantly reduce the time for an anastomotic procedure, less tissue trauma due to less tissue handling, there is early restoration of gastrointestinal function, early resumption of oral feeding and reduced duration of hospital stay which helps ultimately in early return to routine work, importantly staplers can be used at places were hand sewn anastomosis is technically difficult. Due to reduction in operating time, staplers may be advantageous in patients whose general condition is poor and who would not tolerate prolonged anesthesia. HA can be very difficult when access is severely limited especially in low anterior resection; mechanical stapling devices have an added advantage in these situations.

Currently, a titanium (Ti) anastomotic device is the most commonly used. However, Ti staples are not biodegradable, and adverse reactions to the anastomosis are often reported. In addition, the Ti staples produce artifacts on Computed Tomography (CT) and other imaging examination which increase the risk of misdiagnosis. On the contrary, the bioabsorbable staple made from a polymer that can degrade in the human body environment, is an alternative. At present, polylactic and polyglycolic acid subcuticular absorbable staples are available in skin closure. However, the poor mechanical properties of polymers restrain their applications in GI anastomoses which need high closure strength. Therefore, mechanically stronger and biodegradable staples are needed for better anastomoses in the digestive tract.

### SUMMARY

Embodiments of the present disclosure described herein provide bioresorbable zinc-based (i.e., Zn-based) wound closure devices such as, for example, surgical staples, wires, or sutures. In the present disclosure, the Zn-based wound closure devices include Zn and at least one primary alloying element as defined herein below. The Zn-based wound closure devices are biocompatible and biodegradable with good mechanical strength and tissue compatibility. The Zn-based wound closure devices are safe and easy to use during surgical operations and provide shorter recovery and healing time with less chance of clinical complications. Typical applications for Zn-based wound closure devices of the present disclosure include, but are not limited to, skin wound closure, muscular, vascular, and neuronal anastomosis, as well as GI anastomosis and other tissue or organ anastomosis. In certain embodiments, the Zn-based wound closure devices of the present disclosure are used for treatment of the above applications for pediatric patients.

In one aspect of the present disclosure, a bioresorbable wound closure device is provided. In one embodiment of the present disclosure, the wound closure device includes a bioresorbable material composed of zinc (Zn) and at least one primary alloying element, wherein the at least one primary alloying element is selected from the group consisting of chromium (Cr), vanadium (V), and zirconium (Zr).

In some embodiments of the present disclosure, the bioresorbable material can further include at least one secondary alloying element, wherein the at least one secondary alloying element is selected from the group consisting of aluminum (Al), iron (Fe), calcium (Ca), strontium (Sr), silver (Ag), copper (Cu), titanium (Ti), manganese (Mn), selenium (Se), molybdenum (Mo), cobalt (Co), silicon (Si), tin (Sn), nickel (Ni), lithium (Li), sodium (Na), potassium (K), germanium (Ge), rubidium (Rb), tungsten (W), cesium (Ce), scandium (Sc), and yttrium (Y). In some embodiments, the bioresorbable material is devoid of any secondary alloying element.

In some embodiments of the present disclosure, the bioresorbable material is devoid of magnesium (Mg). Mg-containing materials are typically excluded in the present disclosure because such materials have a rapid degradation which does not provide sufficient mechanical support before complete tissue healing takes place, and Mg-containing materials evolve harmful H₂ during degradation, which is a big clinical concern.

In some embodiments of the present disclosure, the at least one primary alloying element, and if present, the at least one secondary alloying element, are present in a non-toxic amount. By “non-toxic amount” it is meant the Zn-based alloy contains a concentration of the primary alloying element (and if present the secondary alloying element) that is not harmful to a mammal when exposed to the same. Zinc itself is typically not harmful to a mammal.

In some embodiments of the present disclosure, the bioresorbable material includes from about 0.1 atomic percent to about 12 atomic percent of the least one primary alloying element.

In some embodiments of the present disclosure, the bioresorbable material includes from about 0.1 atomic percent to about 8 atomic percent of the least one primary alloying element.

In some embodiments of the present disclosure, the bioresorbable material is a binary compound of Zn and Zr.

In some embodiments of the present disclosure, the binary compound includes from about 0.1 atomic percent to about 12 atomic percent Zr, and the remainder of the binary compound material is Zn.

In some embodiments of the present disclosure, the binary compound includes about 0.5 atomic percent Zr.

In some embodiments of the present disclosure, the bioresorbable material is a binary compound of Zn and Cr.

In some embodiments of the present disclosure, the binary compound includes from about 0.1 atomic percent to about 12 atomic percent Cr, and the remainder of the binary compound material is Zn.

In some embodiments of the present disclosure, the binary compound includes about 0.5 atomic percent Cr.

In some embodiments of the present disclosure, the bioresorbable material is a binary compound of Zn and V.

In some embodiments of the present disclosure, the binary compound includes from about 0.1 atomic percent to about 12 atomic percent V, and the remainder of the binary compound material is Zn.

In some embodiments of the present disclosure, the binary compound includes about 0.5 atomic percent V.

In some embodiments of the present disclosure, the at least one primary alloying element is present as an intermetallic phase precipitate in Zn.

In some embodiments of the present disclosure, the at least one primary alloying element forms a local atomic bond with Zn.

In some embodiments of the present disclosure, the bioresorbable material has a compressive yield strength from about 10 MPa to about 1000 MPa.

In some embodiments of the present disclosure, the bioresorbable material has an elastic modulus from about 10 GPa to about 200 GPa.

In some embodiments of the present disclosure, the bioresorbable material has an elongation to failure of from about 1 percent to about 80 percent.

In some embodiments of the present disclosure, the bioresorbable material has a degradation rate of from about 0.01 mm/y to about 1 mm/y.

In some embodiments of the present disclosure, the bioresorbable material is antibacterial.

In some embodiments of the present disclosure, the bioresorbable material exhibits an adhesion for at least one of E. coli and Staphylococcus aureus (S. aureus).

In some embodiments of the present disclosure, the bioresorbable material exhibits an antibacterial rate for E. coli of from about 30 percent to about 100 percent.

In some embodiments of the present disclosure, the bioresorbable material exhibits an antibacterial rate for S. aureus of from about 50 percent to 100 percent.

In some embodiments of the present disclosure, the bioresorbable material is the shape of a staple.

In some embodiments of the present disclosure, the bioresorbable material is implantable into a mammalian body and is configured to close an internal wound.

In some embodiments of the present disclosure, the bioresorbable material is applied on an external surface of a mammalian body and is configured to close an external wound.

In another aspect of the present disclosure, a method of forming a wound closure device is provided. In one embodiment, the method includes mixing, in any order, Zn and at least one primary alloying element to provide a blend of Zn and the at least one primary alloying element, wherein the at least one primary alloying element is selected from the group consisting of Cr, V, and Zr; heating the blend to a temperature that melts at least the least one primary alloying element; cooling the heated blend to provide a bioresorbable material of Zn and the at least one primary alloying element, wherein the bioresorbable material comprises the least one primary alloying element as an intermetallic precipitate in Zn; and shaping the bioresorbable material into a shape of the wound closure device.

In yet a further aspect of the present disclosure, a method of treating a wound of a mammal is provided. In one embodiment of the present disclosure, the method includes applying a wound closure device to a wound of a mammal, the wound closure device including a bioresorbable material composed of Zn and at least one primary alloying element, wherein the at least one primary alloying element is selected from the group consisting of Cr, V, and Zr; and closing the wound with the wound closure device. In one aspect the mammal is a human. In another aspect the mammal is a domestic animal, such as a dog or cat or another animal such as a horse, cow, donkey, or deer. Any mammal subject to wound care and treatment is contemplated by the disclosure.

In some embodiments, the wound is an internal wound, and the applying includes implanting the wound closure device into a body of the mammal. The implantation of the wound closure device may comprise at least one staple in combination with at least one suture or solely at least one staple or solely at least one suture.

In some embodiments of the present disclosure, the wound is an external wound, and the applying includes sewing, suturing or stapling the wound closure device into the skin of the mammal.

### BRIEF DESCRIPTION OF THE DRAWINGS

FIGS. 1A-1H are Scanning Electron Micrograms (SEMs) of pure Zn (FIGS. 1A-1B), Zn-0.5Cr (FIGS. 1C-1D), Zn-0.5Zr (FIGS. 1E-1F) and Zn-0.5V (FIGS. 1G-1H).

FIG. 2 illustrates the XRD patterns of pure Zn, Zn-0.5Cr, Zn-0.5Zr and Zn-0.5V.

FIGS. 3A, 3B and 3C are graphs illustrating mechanical properties of pure Zn, Zn-0.5Cr, Zn-0.5Zr and Zn-0.5V; FIG. 3A shows the ultimate tensile strength (UTS, MPa), FIG. 3B shows the yield strength (YS, MPa), and FIG. 3C shows the elongation rate to failure (ER, %).

FIG. 4 is a graph illustrating the corrosion rates (CR_{w}) of pure Zn, Zn-0.5Cr, Zn-0.5Zr and Zn-0.5V.

FIG. 5 is a graph illustrating the pH change for pure Zn, Zn-0.5Cr, Zn-0.5Zr and Zn-0.5V.

FIG. 6 is a graph illustrating the MTT assay (a colorimetric assay for assessing cell metabolic activity) for cell viability of endothelial cells for a control sample of cells culture medium only without any materials, pure Zn, Zn-0.5Cr, Zn-0.5Zr and Zn-0.5V after 1 day and 3 days.

FIG. 7 is a graph illustrating the Zn ion concentration in the corresponding extract.

FIGS. 8A-8H are SEMs showing the endothelial cell adhesion morphology when cultured on pure Zn (FIGS. 8A-8B), Zn-0.5Cr (FIGS. 8C-8D), Zn-0.5Zr (FIGS. 8E-8F) and Zn-0.5V (FIGS. 8G-8H), respectively for 3 days.

FIGS. 9A-9H are SEMs showing platelet adhesion morphology when cultured on pure Zn (FIGS. 9A-9B), Zn-0.5Cr (FIGS. 9C-9D), Zn-0.5Zr (FIGS. 9E-9F) and Zn-0.5V (FIGS. 9G-9H), respectively for 24 hours.

FIG. 10 is a graph illustrating the number of adhered platelets cultured on Zn, Zn-0.5Cr, Zn-0.5Zr and Zn-0.5V for 24 hours.

FIG. 11 is a graph illustrating the hemolysis percentage compared between groups.

FIGS. 12A-12D are SEMs of E. coli adhesion on pure Zn (FIG. 12A), Zn-0.5Cr (FIG. 12B), Zn-0.5Zr (FIG. 12C) and Zn-0.5V (FIG. 12D).

FIGS. 13A-13D are SEMs of S. aureus adhesion on pure Zn (FIG. 13A), Zn-0.5Cr (FIG. 13B), Zn-0.5Zr (FIG. 13C) and Zn-0.5V (FIG. 13D).

FIGS. 14A and 14B are graphs illustrating the antibacterial rates of the culture medium after culturing with E. coli (FIG. 14A), and with S. aureus (FIG. 14B); a control of culture medium only without any materials, pure Zn, Zn-0.5Cr, Zn-0.5Zr and Zn-0.5V are exemplified.

FIGS. 15A and 15B are graphs illustrating the Zn ion concentration in the culture medium after culturing with E. coli (FIG. 15A), and with S. aureus (FIG. 15B) of a control of culture medium only without any materials, pure Zn, Zn-0.5Cr, Zn-0.5Zr and Zn-0.5V; a control, pure Zn, Zn-0.5Cr, Zn-0.5Zr and Zn-0.5V are exemplified.

### DETAILED DESCRIPTION

The present disclosure will now be described in greater detail by referring to the following discussion and drawings that accompany the present disclosure. In the following description, numerous specific details are set forth, such as particular structures, components, materials, dimensions, processing steps and techniques, in order to provide an understanding of the various embodiments of the present disclosure. However, it will be appreciated by one of ordinary skill in the art that the various embodiments of the present disclosure may be practiced without these specific details. As used throughout the present disclosure, the term “about” generally indicates no more than +10%, +5%, +2%, +1% or +0.5% from a number. When a range is expressed in the present disclosure as being from one number to another number (e.g., 20 to 40), the present disclose contemplates any numerical value that is within the range (i.e., 22, 24, 26, 28.5, 31, 33.5, 35, 37.7, 39 or 40) or any in amount that is bounded by any of the two values that can be present within the range (e.g., 28.5-35).

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising”, when used in this disclosure, specify the presence of stated features, integers, steps, operations, elements, and/or components, but do not preclude the presence or addition of one or more other features, integers, steps, operations, elements, components, and/or groups thereof.

As mentioned above, Ti surgical staples are the dominating staples used in surgeries. The applications of these staples in anastomoses shorten the operation time, reduce surgical complication and alleviate the patient's pain, and strong wound closure. Ti surgical staples have sufficient mechanical strength and ductility for staple applications, but are not biodegradable. Their life-long presence may interfere with imaging examinations and cause clinical complications such as chronic inflammation, bleeding and infection. The most significant drawbacks of Ti staples are anastomotic leakage and stricture. Among the approximately 3 million GI anastomoses each year in the USA using Ti staples, up to 15% cases had leakage, stricture and other complications. Anastomotic leakage is a significant cause of early post-operative morbidity that may lead to re-operation, prolonged hospital stays, psychological trauma and death. Anastomotic stricture can result in post-operative dysphagia, which may require additional invasive procedures with increased frequency of outpatient attendance and overall cost as well as nutritional compromise and reduction in quality of life. Ti staples also elicited significant foreign body reaction in human GI anastomoses. Another significant deterring factor for permanent Ti implant placement in children is the impending growth. A second revision surgery must be performed for removal. Collectively, Ti staple might not be a good choice, especially for pediatric applications where a bioresorbable Zn staple has the potential to be a smarter alternative.

Biodegradable polymeric staples made of polylactic and polyglycolic acid are quite successful in subcuticular applications. Polymeric stables offer lower incidence of infection, reduced operative time, less patient discomfort and decreased analgesic use, and excellent cosmetic result. However, there are some applications where the polymeric staples were found unsuitable. For instance, polymeric staples were deemed insufficient in strength to retain revascularization edema that resulted in an open wound and to a higher-than normal wound infection rate in peripheral vascular surgical procedures. The polymeric stapler was also found difficult to use in some firm tissues which prevented the stapler from firing, and the tissue capture necessary for staple deployment was not achieved. Moreover, the poor mechanical properties of polymeric staples restrain their applications in GI anastomosis which need high closure strength. These identified inadequacies in the properties of the polymer staples, thereby, opens opportunities for other types of biodegradable materials, e.g., biodegradable metals.

Due to the biodegradable property, good biocompatibility, and significantly higher mechanical stability and ductility than polymers, Mg and its alloys have been considered to be candidates as surgical staples. For example, high purity Mg staples were shown to exhibit good biocompatibility with minimal inflammation in small intestine anastomosis of minipigs and in gastric anastomosis in domestic pigs. Nonetheless, two critical weaknesses are associated with Mg staples: 1) too rapid degradation to provide sufficient mechanical support before a complete tissue healing takes place, and 2) harmful H₂ evolution during degradation, a big clinical concern. In GI anastomoses where the staples are exposed to high acidic gastric juice or alkaline intestinal juice, higher-than-normal degradation speed is expected for the staples. Mg staples, thereby suffer even faster degradation, making them unsuitable for GI anastomoses due to premature loss of mass and mechanical integrity to support a complete wound healing. For subcuticular applications, Mg degradation comes with concerns regarding H₂ gas evolution. Since the staple is deployed below the skin, gas packets could form that can result in tissue swelling and dehiscence.

The present disclosure provides an alternative material besides Ti, polymeric or Mg that can be used in providing wound closure devices. The term “wound closure device” is used throughout the present disclosure to denote a device that is capable of closing an internal and/or external wound. Notably, the present disclosure provides wound closure devices, such as, for example, staples, wires and sutures, which are composed of a bioresorbable material composed of zinc (Zn) and at least one primary alloying element. The at least one primary alloying element is selected from the group consisting of Cr, V, and Zr. The “bioresorbable material” is used throughout the present application to denote materials that degrade safely within the body of a mammalian species. The Zn-based wound closure devices of the present disclosure overcome many of the above noted drawbacks that are associated with Ti, polymeric and Mg staples. Notably, the Zn-based wound closure devices of the present application, arc biocompatible, biodegradable, bioresorbable, non-toxic, have good mechanical properties, have degradation rates that provide sufficient mechanical support to allow complete tissue healing to take place, and no harmful H₂ evolution is observed during degradation.

The use of the Zn-based bioabsorbable material of the present disclosure as alternative biodegradable wound closure devices offers some distinct advantages. For example, Zn is stronger than its polymeric counterparts, and therefore, is most suitable in the applications where the strength of the polymer staple was inadequate (e.g., for closing surgical wounds after a peripheral vascular surgical or GI tract procedure). Moreover, the higher strength of Zn affords a redesign of the staple. Notably, and in certain embodiments, the Zn-based staple is thinner and has a smaller footprint than conventional staples. In some embodiments, the Zn-based staple of the present disclosure has a thickness from about 0.1 mm to about 10 mm, with a thickness from about 0.5 mm to about 2 mm being more typical. A small staple is suitable for closing small and tight wounds. A strong and thin staple allows the possibility of gripping tissues where skin tension is high and in sections where subcuticular space is small. Furthermore, the relatively high hardness of Zn staples would allow it to pierce thick tissues easily and hence would extend the applicability of surgical staples to other fields (e.g., veterinary medicine). Unlike Ti, Zn is bioresorbable, thus no removal surgery is needed, and it won't impede growth or cause chronic inflammation, bleeding and infection. The bioresorbable nature also minimizes the chance of anastomotic leakage and stricture thanks to the disappearance of the implants.

Zn is one of the most abundant essential elements in the human body, mostly in muscles and bones. Zn plays important roles in the structure and function of over 300 enzymes and other macromolecules like Zn-finger proteins. It should be noted that the risk of systemic Zn toxicity associated with Zn alloys is negligible. In certain embodiments, the Zn-based wound closure devices of the present disclosure include several hundred mg of pure metal. Assuming complete degradation within 12 months, the expected daily dose of Zn is less than 1 mg/day, far below the recommended maximum daily intake value of 10 mg/day. In vivo animal studies also demonstrated good biosafety of Zn implants without any systemic toxicity reported so far. Furthermore, Zn exhibits good biocompatibility to promote various tissue regenerations around the implants. This feature adds significantly to the possible health benefits of a bioresorbable Zn implant.

As stated above, the preset disclosure provides a wound closure device that includes a bioresorbable material composed of zinc (Zn) and at least one primary alloying element, wherein the at least one primary alloying element is selected from the group consisting of Cr, V, and Zr. In some embodiments, a single primary alloying element (i.e., Cr, V or Zr) can be employed. In another embodiment, two primary alloying elements (e.g., Cr—V, V—Zr, or Cr—Zr) can be employed. In yet another embodiment, all three listed primary alloying elements (i.e., Cr, V and Zr) can be employed.

In accordance with the present disclosure, the at least one primary alloying element is present in a non-toxic amount, as defined previously herein. In some embodiments of the present disclosure, the bioresorbable material includes from about 0.1 atomic percent to about 12 atomic percent of the least one primary alloying element; the remainder of the bioresorbable material can be Zn itself or a combination of Zn and at least one secondary alloying element as will be defined herein below. In other embodiments of the present disclosure, the bioresorbable material includes from about 0.1 atomic percent to about 8 atomic percent of the least one primary alloying element; the remainder of the bioresorbable material can be Zn itself or a combination of Zn and at least one secondary alloying element as will be defined herein below. In yet other embodiments of the present disclosure, the bioresorbable material includes from about 0.1 atomic percent to about 3 atomic percent of the least one primary alloying element; the remainder of the bioresorbable material can be Zn itself or a combination of Zn and at least one secondary alloying element as will be defined herein below.

In some embodiments, and in addition to Zn and at least one of the primary alloying elements, the bioresorbable material can further include at least one secondary alloying element. In the present disclosure, the at least one secondary alloying element is selected from the group consisting of Al, Fe, Ca, Sr, Ag, Cu, Ti, Mn, Se, Mo, Co, Si, Sn, Ni, Li, Na, K, Ge, Rb, W, Ce, Sc, and Y. In some embodiments, the at least one secondary alloying element is one of Al, Fc, Ca, Na or K.

In accordance with the present disclosure, the at least one secondary alloying element is present in a non-toxic amount, as defined previously herein. In some embodiments of the present disclosure, the bioresorbable material includes from about 0.1 atomic percent to about 15 atomic percent of the least one secondary alloying element; the remainder of the bioresorbable material is a combination of Zn and at least one primary alloying element. In other embodiments of the present disclosure, the bioresorbable material includes from about 0.1 atomic percent to about 12 atomic percent of the least one secondary alloying element; the remainder of the bioresorbable material is a combination of Zn and at least one primary alloying element. In yet other embodiments of the present disclosure, the bioresorbable material includes from about 0.1 atomic percent to about 10 atomic percent of the least one secondary alloying element; the remainder of the bioresorbable material is a combination of Zn and at least one primary alloying element.

In any of the embodiments mentioned above. Mg is excluded from the bioresorbable material of the present disclosure. Mg is excluded in the present disclosure because such Mg-containing bioresorbable materials have a rapid degradation which does not provide sufficient mechanical support before a complete tissue healing takes place, and Mg-containing bioresorbable materials evolve harmful H₂ during degradation, which is a big clinical concern. Although Mg is generally excluded from the bioresorbable material of the present disclosure, and in cases in which rapid degradation and/or ‘H₂’ evolution are not a concern, Mg can be used as one of the secondary alloying elements.

In some embodiments of the present disclosure, the bioresorbable material is a binary compound. The term “binary compound” is used throughout the present disclosure to denote that the bioresorbable material includes two elements, that first of which is Zn and the second of which is at least one of the primary alloying elements (without any secondary alloying element present). In one embodiment, the binary compound is composed of Zn and Zr. In another embodiment, the binary compound is composed of Zn and V. In yet another embodiment, the binary compound is composed of Zn and Cr. In such embodiments, the binary compound includes from about 0.1 atomic percent to about 12 atomic percent, more preferably from about 0.1 atomic percent to about 8 atomic percent, and even more preferably, from about 0.1 atomic percent to about 3 atomic percent of the least one primary alloying element, the remainder of the binary compound is Zn. In one highly preferred embodiment, the binary compound includes about 0.5 atomic percent of the at least one primary alloying element (Cr, V, or Zr), and the remainder is Zn.

The bioresorbable material that provides the wound closure device of the present disclosure can be formed by first mixing (i.e., blending) Zn and at least one of the primary alloying elements and optionally at least one of the secondary alloying elements. The constituents that provide the bioresorbable material can be added in any order. For example, at least one of the primary alloying elements can be added to Zn, and thereafter, and if present, the at least one secondary alloying element can be added to the Zn-primary alloying element addition. Mixing can occur during each addition step or a single mixing step can be employed after each of the constituents that provide the bioresorbable material are added together. This mixing step can be performed utilizing any conventional mixing apparatus. The mixing step can provide a homogenous blend of Zn, at least one primary alloying element, and, optionally, the at least one secondary alloying elements.

After providing the blend, the blend is heated to a temperature that melts at least the at least one first alloying element that is present in the blend. This heating step can be performed in any conventional heating apparatus including, for example, an oven or furnace. In one example, the temperature of the heating step is from about 100° C. to about 600° C. The heating step can be performed for various time periods. In one example, the heating step is performed for a time period from about 10 minutes to 2000 minutes. The heating step is typically performed under vacuum or in an inert ambient such as, for example, helium, argon and/or neon.

After performing the heating step, the heated mixture is cooled so as to provide a bioresorbable material in which the at least one primary alloying element is present as an intermetallic phase precipitate in Zn. When a secondary alloying element is present, the secondary alloying element can also be present as an intermetallic phase precipitate. By “intermetallic phase precipitate” it is meant the basic structure and distribution of the phases alter on normal thermal exposures. The intermetallic phase precipitate provides a bioresorbable material that has enhanced mechanical strength and physical properties as compared to a bioresorbable material in which no intermetallic phase precipitate is present. Cooling can be performed by water quenching. Cooling can be a rapid cooling from the highest temperature used during the heating step back to ambient room temperature, i.e., 20° C. to 30° C. The cooling can be performed at a cooling rate of from about 1° C./min to about 100° C./min.

In the present disclosure, and after cooling, the primary alloying element forms a local atomic bond with Zn. By “local atomic bond” it is meant that the chemical bonding responsible for the interactions between atoms and molecules. The formation of local atomic bonds further enhances the mechanical strength of the bioresorbable material of the present disclosure.

After cooling, the resultant bioresorbable material of Zn and the at least one primary alloying element, and optionally, the at least one secondary alloying element can be formed into a wound closure device having a desirable shape utilizing techniques that are well-known to those skilled in the art. For example, extrusion or cast molding can be used in the present disclosure to provide a surgical staple, wiring or suture.

In embodiments of the present disclosure, the bioresorbable material (and thus the wound closure device) contains crystals of the first alloying element that have a crystal size from about 0.1 μm to about 2000 μm, with a crystal size from about 1 μm to about 100 μm being more typical. The bioresorbable material (and thus the wound closure device) has an equiaxed α-Zn phase with uniformly distributed second phases composed of at least the primary alloying element. The bioresorbable material (and thus the wound closure device) can have a grain size from about 0.1 μm to about 2000 μm, with a grain size from about 1 μm to about 100 μm being more typical. The bioresorbable material (and thus the wound closure device) has a hexagonal close-packed structure. The Zn-based bioresorbable material of the present disclosure has mechanical and physical properties which are a closer match to mammalian bone than Ti alloys, Co—Cr alloys or stainless steel (SS). When present in an acidic environment in the stomach, the Zn-based bioresorbable material of the present disclosure has an accelerated degradation rate.

In some embodiments of the present disclosure, the bioresorbable material (and thus the wound closure device) has a compressive yield strength from about 10 MPa to about 1000 MPa, with a compressive yield strength from about 200 MPa to about 500 MPa being more typical. In the present disclosure, the compressive yield strength determines the maximum allowable load in a mechanical component, since it represents the upper limit to forces that can be applied without producing permanent deformation. Compressive yield strength is measured in the present disclosure by compression mechanical test through a Stress-strain curve analysis.

In some embodiments of the present disclosure, the bioresorbable material (and thus the wound closure device) has an elastic modulus from about 10 GPa to about 200 GPa, with an clastic modulus from about 100 GPa to about 150 GPa being more typical. In the present disclosure, the elastic modulus determines the material's ability to resist deformation and return to its original shape after the force is removed. Elastic modulus is measured in the present disclosure by mechanical test through a Stress-strain curve analysis.

In some embodiments of the present disclosure, the bioresorbable material (and thus the wound closure device) has an elongation to failure of from about 1 percent to about 80 percent, with an elongation to failure of from about 5 percent to about 30 percent being more typical. In the present disclosure, the elongation to failure determines the material's ductility or its ability to stretch before it breaks. Elongation to failure is measured in the present disclosure by mechanical test through a Stress-strain curve analysis.

In some embodiments of the present disclosure, the bioresorbable material (and thus the wound closure device) has a degradation rate of from about 0.01 mm/y to about 1 mm/y, with a degradation rate of from about 0.1 mm/y to about 0.5 mm/y being more typical. In the present disclosure, the degradation rate determines the material's ability to degrade or deteriorate over time when exposed to certain environmental conditions. Degradation rate is measured in the present disclosure by weight loss in the immersion tests and volume loss in the in vivo animal implantation tests.

In some embodiments of the present disclosure, the bioresorbable material (and thus the wound closure device) is antibacterial, i.e., it is effective against bacteria. In the present disclosure, the bioresorbable material exhibits an adhesion for bacteria. In one example, the bioresorbable material of the present disclosure exhibits an adhesion for E. coli. In another example, the bioresorbable material of the present disclosure exhibits an adhesion for S aureus.

In some embodiments of the present disclosure, the bioresorbable material (and thus the wound closure device) exhibits an antibacterial rate for E. coli of from about 30 percent to about 100 percent, with antibacterial rate for E. coli of from about 50 percent to about 99.99 percent being more typical. In the present disclosure, the antibacterial rate for E. coli can be measured by a spread plate method and bacterial morphology.

In some embodiments of the present disclosure, the bioresorbable material (and thus the wound closure device) exhibits an antibacterial rate for S. aureus of from about 50 percent to 100 percent, with antibacterial rate for S. aureus of from about 90 percent to about 99.99 percent being more typical. In the present disclosure, the antibacterial rate for S. aureus can be measured by a spread plate method and bacterial morphology.

In some embodiments of the present disclosure, the bioresorbable material (and thus the wound closure device) is implantable into a mammalian body and is configured to close an internal wound. The term “mammalian” includes humans and non-humans such as, for example, rats, cats, dogs, horses, etc. Examples of an internal wound include, but are not limited to, internal fractures, surgical wounds, or organ damage. In other embodiments of the present disclosure, the bioresorbable material (and thus the wound closure device) is applied on an external surface of a mammalian body and is configured to close an external wound. Examples of an external wound include, but are not limited to, puncture wounds, abrasions, cuts, or lacerations. The wound closure devices of the present disclosure can be applied to the internal wound and/or external wound utilizing techniques that are well known in the art.

The wound closure device of the present disclosure can be used in a method of treating a wound of a mammal. In one embodiment of the present disclosure, the method includes applying the wound closure device of the present disclosure to a wound of a mammal; and closing the wound with the wound closure device. The applying of the wound closure device depends on the exact type of wound closure device, i.e., staple, wire, or suture, used and it includes techniques well known in the art. The closing of the wound also in dependent on the type of wound closure device used and it includes techniques well known in the art. In some embodiments, the wound is an internal wound, and the applying includes implanting the wound closure device into a body of the mammal. The implanting can include sewing or stapling. In some embodiments of the present disclosure, the wound is an external wound, and the applying includes sewing or stapling the wound closure device into the skin of the mammal.

Examples have been set forth below for the purpose of further illustrating the present disclosure. The scope of this disclosure is not to be in any way limited by these examples.

### Example 1: Bioresorbable Material Preparation

Zn binary alloy ingots blended with different alloying elements were prepared from pure Zn (99.99%), pure Cr (99.99%), pure Zr (99.99%), and pure V (99.9%) ingots by gravity casting. The casted cylinders were heat-treated at 350° C. for 48 hrs and water quenched and further extruded at 260° C. from Ø 28 mm to Ø 10 mm. The extruded alloys were labeled as Zn-0.5V, Zn-0.5Cr, and Zn-0.5Zr alloys. Here, the 0.5 denotes atomic percent of the primary alloying element that is present in the Zn alloy. Pure Zn (99.99%) was also extruded as a comparison. All the Zn materials were cut into discs (Ø 10 mm×5 mm) for in vitro tests or drawn to thin wires (Ø 0.25 mm) for in vivo tests. The disc samples were polished using #1200 sandpaper, while the wire samples were electropolished using a voltage of 10V in a mixture of ethanol (885 ml), butanol (100 ml), aluminum chloride hexahydrate (AlCl₃·6H₂O) (109 g), zinc chloride (ZnCl₂) (250 g) and water (120 ml) for 2 min.

### Example 2: Microstructure of Bioresorbable Materials

Optical micrographs and XRD patterns of the extruded Zn and its alloys were characterized. Notably, the microstructure and phase composition of the samples prepared in Example 1 (i.e., Zn-0.5V, Zn-0.5Cr, Zn-0.5Zr, and pure Zn) were characterized using optical microscopy (Olympus BX51M) and X-ray diffraction (XRD, Rigaku DMAX240), respectively. The XRD was equipped with Cu Ka radiation and operated at 40 kV and 100 mA with the scanning rate and step of 4 degrees/min and 0.02 degrees.

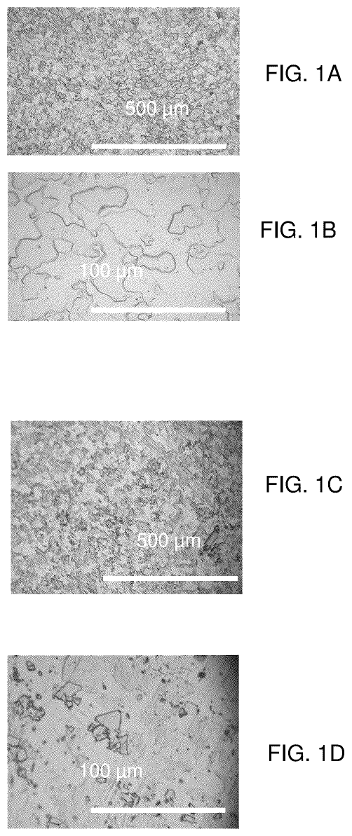

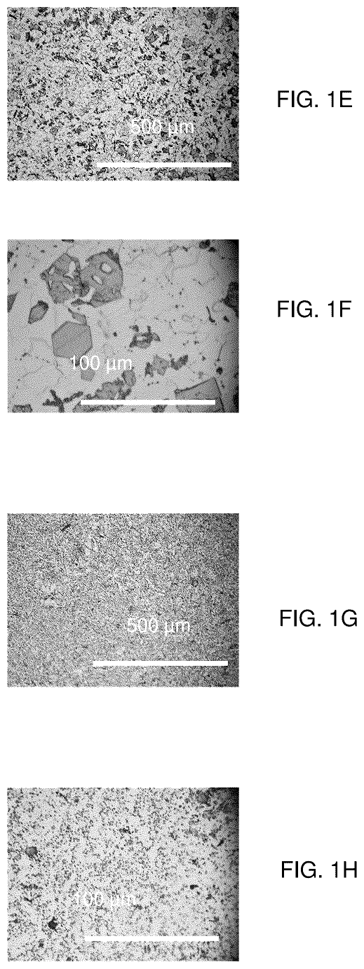

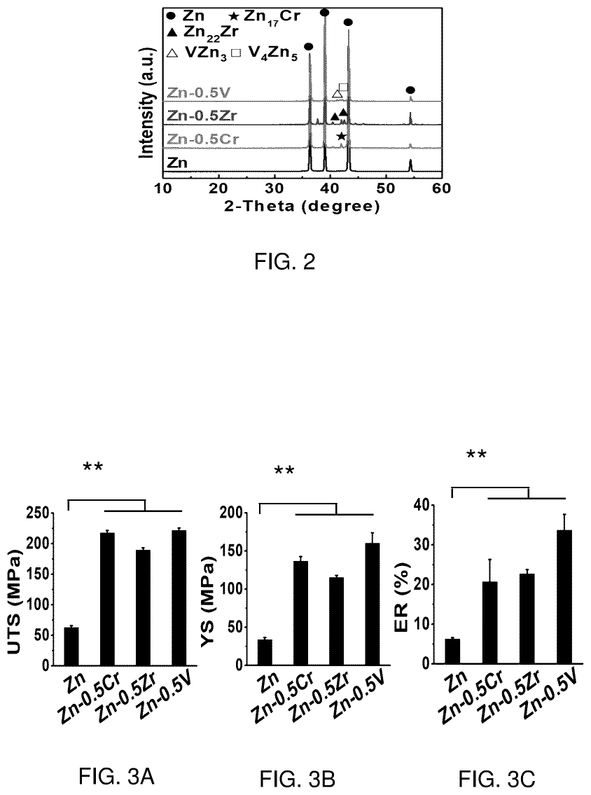

All the Zn materials including pure Zn (FIGS. 1A-1B), Zn-0.5Cr (FIGS. 1C-1D), Zn-0.5Zr (FIGS. 1E-1F) and Zn-0.5V (FIGS. 1G-1H) showed similar crystal sizes, and all the alloys showed an equiaxed α-Zn phase with uniformly distributed second phases, see FIGS. 1A-1H. Especially, the Zn-0.5V alloy (FIGS. 1G and 1H) exhibited a much finer grain size and second phase structure when compared to the other two alloys (FIGS. 1C-1F). The XRD patterns shown in FIG. 2 indicated that all the alloys showed a similar hexagonal close-packed structure with different second phases for different alloys.

### Example 3: Mechanical Properties of Bioresorbable Materials

The samples prepared in Example 1 including pure Zn, Zn-0.5Cr, Zn-0.5Zr and Zn-0.5V were machined along the extrusion direction for the mechanical test according to ASTM-E8/E8M standards (See, A.A.S.f. Testing, Materials, Standard test methods for tension testing of metallic materials, ASTM international 2009). The test was carried out on a universal material test machine (Instron 5969, USA) at a strain rate of 1×10⁻⁴ s⁻¹. The yield strength was determined as the stress at which the 0.2% plastic deformation occurred.

Compared to pure Zn, the Cr, V and Zr alloys significantly improved the mechanical strengths to around 200 MPa (ultimate tensile strength, UTS) and 120-160 MPa (yield strength, YS), while the elongations were also significantly increased to 20-30% simultaneously (See, for example, FIGS. 3A, 3B and 3C). The Zn-0.5V alloy possessed the best combination of mechanical strength and ductility among all groups. The fracture morphology also indicated similar trends. The pure Zn exhibited a smooth fracture surface without obvious plastic deformation, while the Zn alloys showed a quasi-cleavage fracture morphology with numerous cleavage planes. Many dimples appeared on the fracture surface of Zn-0.5V alloy, indicating its excellent plastic deformation ability.

### Example 4: In Vitro Degradation of Bioresorbable Materials

All the degradation tests of the samples prepared in Example 1 were carried out in a modified Hanks' solution at 37±0.5° C. as described previously; see, for example, Levesque, H. Hermawan, D. Dube, D. Mantovani, Design of a pseudo-physiological test bench specific to the development of biodegradable metallic biomaterials, Acta Biomater. 4 (2) (2008) 284-95. Y. Su, S. Champagne, A. Trenggono, R. Tolouei, D. Mantovani, H. Hermawan, Development and characterization of silver containing calcium phosphate coatings on pure iron foam intended for bone scaffold applications, Mater Design 148 (2018) 124-134, Z. M. Shi, A. Atrens, An innovative specimen configuration for the study of Mg corrosion, Corros. Sci. 53 (1) (2011) 226-246, or Y. Su, G. Li, J. Lian, A Chemical Conversion Hydroxyapatite Coating on AZ60 Magnesium Alloy and Its Electrochemical Corrosion Behaviour, Int. J. Electrochem. Sci. 7 (11) (2012) 11497-11511.

The immersion degradation tests were performed for 1 and 3 months according Y. Su, S. Champagne, A. Trenggono, R. Tolouei, D. Mantovani, H. Hermawan, Development and characterization of silver containing calcium phosphate coatings on pure iron foam intended for bone scaffold applications. Briefly, the solution was refreshed every week due to the slow degradation of Zn materials. The pH values of the solution with Zn and its alloys were monitored during the immersion tests. The surface morphologies and phase composition of Zn and its alloys after 1 and 3 months of immersion were characterized using a scanning electron microscope (SEM) and XRD. The CR_{w} (mm/y) was calculated based on weight loss (Wₗₒₛₛ, mg) according to the following equation (see, for example, A. G31-12a, Standard Guide for Laboratory Immersion Corrosion Testing of Metals, ASTM West Conshohocken, PA, 2012):

$$C {R}_{W} = 87.6 \times \frac{{W}_{loss}}{ρ At}$$

where ρ is the material density (g/cm³), t is immersion time (h), A is the sample surface area before the immersion test (cm²). It is noteworthy that the surface area change over time was not considered.

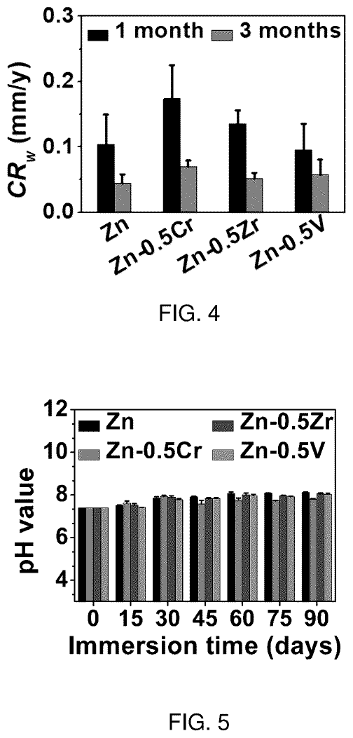

The degradation behaviors were characterized by the degraded morphology, degradation products, corrosion rate (FIG. 4), and pH change (FIG. 5). At one month of immersion, there was similar corrosion product morphology on the pure Zn and Zn-0.5Zr and Zn-0.5V alloys with the formation of a fine-structured layer made of nanorods clusters, while a porous and cracked corrosion products layer formed on the Zn-0.5Cr surface. Correspondingly, the corrosion rate of Zn-0.5Cr was higher than the other groups. XRD indicated that the main degradation products were similar on these samples, which consisted of Zn(OH)₂ and CaZn₂(PO₄)₂·2H₂O. After three months of immersion, the pure Zn and Zn-0.5Cr alloy had severe corrosion morphology with loosely packed corrosion products, but deep corrosion pits were observed on pure Zn. There were some flake-like corrosion products formed on the surfaces of Zn-0.5Zr and Zn-0.5V alloys, which correspond to Zn₃(PO₄)₂·4H₂O and CaZn₂(PO₄)₂·2H₂O. All the samples showed similar corrosion rates and stable pH change during the three months of immersion (see, for example, FIGS. 4-5).

### Example 5: In Vitro Biocompatibility of Bioresorbable Materials

### Hemocompatibility

The hemolysis and platelet adhesion tests were performed according to the method described previously; see, for example, H. Yang, X. Qu, W. Lin, C. Wang, D. Zhu, K. Dai, Y. Zheng, In vitro and in vivo studies on zinc-hydroxyapatite composites as novel biodegradable metal matrix composite for orthopedic applications, Acta Biomater. 71 (2018) 200-214, and X. Gu, Y. Zheng, Y. Cheng, S. Zhong, T. Xi, In vitro corrosion and biocompatibility of binary magnesium alloys, Biomaterials 30 (4) (2009) 484-98. In brief, healthy human blood (Zen-Bio, US) was diluted by 0.9% sodium chloride solution with a volume ratio of 4:5. Zinc and its alloys were pre-treated with 9.8 ml 0.9% sodium chloride solution at 37° C. for 30 min. 0.2 mL diluted blood was then added to each tube and incubated at 37° C. for 60 min. Deionized water and 0.9% sodium chloride solution were incubated with 0.2 mL diluted blood as positive and negative control. After centrifuging at 3,000 rpm for 5 min, the supernatants were transferred into 96-well plates and the absorbance (A) was measured by a plate reader (Cytation 5, Biotek, US) at 545 nm. The hemolysis rate (HR) was calculated by the following equation: Hemolysis=(Aₛₐₘₚₗₑ−A_{negative})/(Aₚₒₛᵢₜᵢᵥₑ−A_{negative}).

Platelet-rich plasma (PRP) (Zen-Bio, US) was used for the platelet adhesion test. 50 μl PRP containing 10⁸/μl platelets was overlaid on Zn samples and incubated at 37° C. for 1 hour. After gently rinsing with PBS three times to remove the non-adherent platelets, adherent platelets on samples were fixed with 4% paraformaldehyde (PFA, Affymetrix, U.S.) and 2% glutaraldehyde solution (Fisher Chemical, U.S.) at room temperature for two hours. Samples were then dehydrated with a gradient alcohol solution (30%, 50%, 70%, 90%, and 100%) and hexamethyldisilazane (HMDS) for 10 min, respectively, before they were dried in a desiccator. The samples were sputtered with gold and observed by SEM. At least five different SEM images were selected for counting the number of adherent platelets on each sample.

### Cytocompatibility

Human endothelial cells (EA.hy926, ATCC CRL-2922, US) were cultured in 75 cm² flask (BD Bioscience) with Dulbecco's Modified Eagle Medium (DMEM, ATCC, US) containing 10% fetal bovine serum (FBS, ScienCell) and 1% penicillin/streptomycin solution (P/S, ScienCell) (see, for example, S. Hauser, F. Jung, J. Pietzsch, Human Endothelial Cell Models in Biomaterial Research, Trends Biotechnol. 35 (3) (2017) 265-277). Indirect MTT assay (Thermo Fisher Scientific, US) was used to measure the cell viability with extracts prepared by incubating Zn samples in the culture media at a ratio of 1.25 cm²/mL for 3 days. The Zn ion concentrations in the collected extracts were measured using a Zn colorimetric assay kit (Bio-Vision, US). The extracts were diluted with culture media to specific concentrations of 25% (see, for example, Y. Su, K. Wang, J. Gao, Y. Yang, Y. X. Qin, Y. Zheng, D. Zhu, Enhanced cytocompatibility and antibacterial property of zinc phosphate coating on biodegradable zinc materials, Acta Biomater. 98 (2019) 174-185. J. Fischer, D. Pröfrock, N. Hort, R. Willumcit, F. Feyerabend, Improved cytotoxicity testing of magnesium materials, Materials Science and Engineering: B 176 (11) (2011) 830-834, or J. Wang, F. Witte, T. Xi, Y. Zheng, K. Yang, Y. Yang, D. Zhao, J. Meng, Y. Li, W. Li, K. Chan, L. Qin, Recommendation for modifying current cytotoxicity testing standards for biodegradable magnesium-based materials, Acta Biomater. 21 (2015) 237-49). The cell viability was measured after cultured with the extract solution for 1, 3, and 5 days. Cells with a density of 1×10⁵/well were seeded onto each Zn sample in a 24 well plate. After three days of cell culture, the cell morphology was observed by SEM after fixation and dehydration in the same method described above.

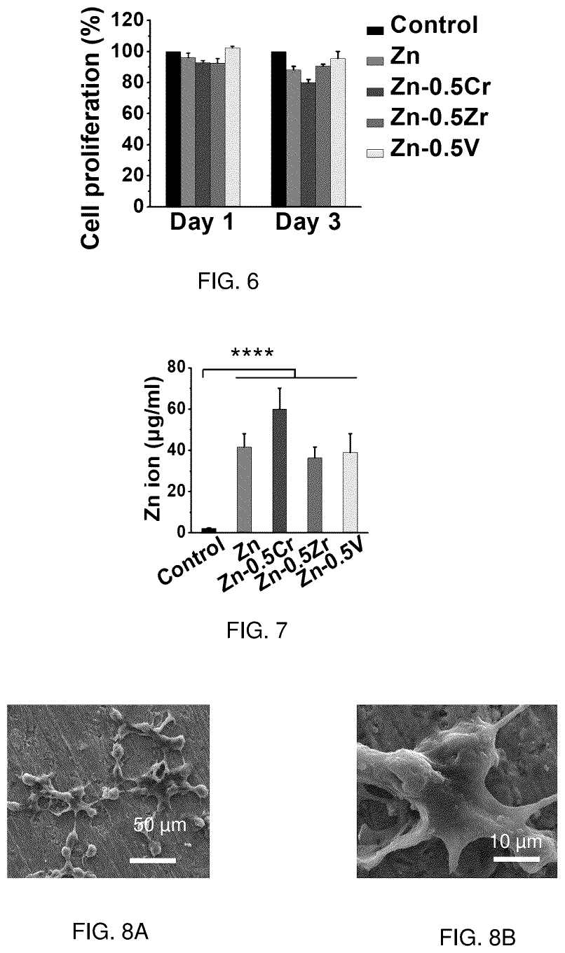

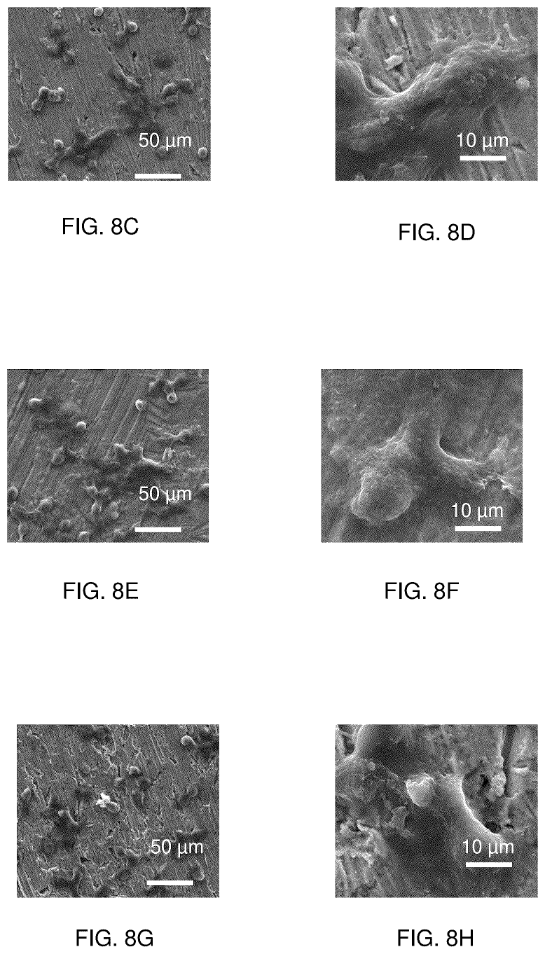

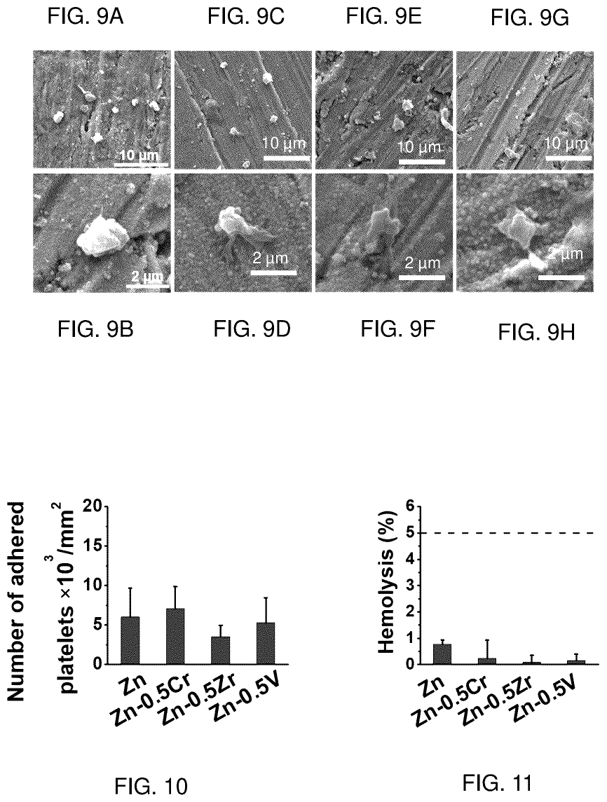

Endothelial cell viability and adhesion, platelet adhesion, and hemolysis tests were conducted to evaluate the cytocompatibility and hemocompatibility of Zn and its alloys. The endothelial cells in all Zn alloy groups showed similar cell viability but better attachment and adhesion when compared to pure Zn group (see, FIGS. 6 and 8A-8D) The Zn ion releases from all Zn samples were similar due to their similar degradation rates (see, FIG. 7). A few adhered platelets were dispersedly distributed on all the Zn surfaces with little spreading (see, FIGS. 8A-8H). The numbers of adhered platelets on all Zn surfaces were similar (see, FIGS. 9A-9H), and their hemolysis rates were also much lower than the hemolysis limit (5%) (see, FIGS. 10-11), indicating their good hemocompatibility.

### Example 6: Antibacterial Performance of Bioresorbable Materials

Escherichia coli (E. coli, ATCC 25922, U.S.) and Staphylococcus aureus (S. aureus, ATCC 29213, U.S.) were cultured according to the procedures from D. A. Robinson, R. W. Griffith, D. Shechtman, R. B. Evans, M. G. Conzemius, In vitro antibacterial properties of magnesium metal against Escherichia coli, Pseudomonas aeruginosa and Staphylococcus aureus, Acta Biomater. 6 (5) (2010) 1869-77. Briefly, the bacteria were cultured in Tryptic Soy Broth (TSB) media at 37° C. and 220 rpm to reach the optical density of 0.5-0.6 at 600 nm. The antibacterial performance was tested with the same procedures from Y. Su, K. Wang, J. Gao, Y. Yang, Y. X. Qin, Y. Zheng, D. Zhu, Enhanced cytocompatibility and antibacterial property of zinc phosphate coating on biodegradable zinc materials, Acta Biomater. 98 (2019) 174-185. 2 ml of the diluted bacterial suspension with a 5×10⁵/mL concentration in TSB media was incubated with samples for 24 hrs at 37° C. and 120 rpm. Diluted bacterial suspension without samples was used as the negative control. The absorbance of the collected bacteria suspension was read at 600 nm. Antibacterial rates in the TSB media were calculated with the following equation: Antibacterial rates=(A_{negative}−Aₛₐₘₚₗₑ)/A_{negative}. Before the SEM imaging, samples were fixed and dehydrated with the same procedures as described above.

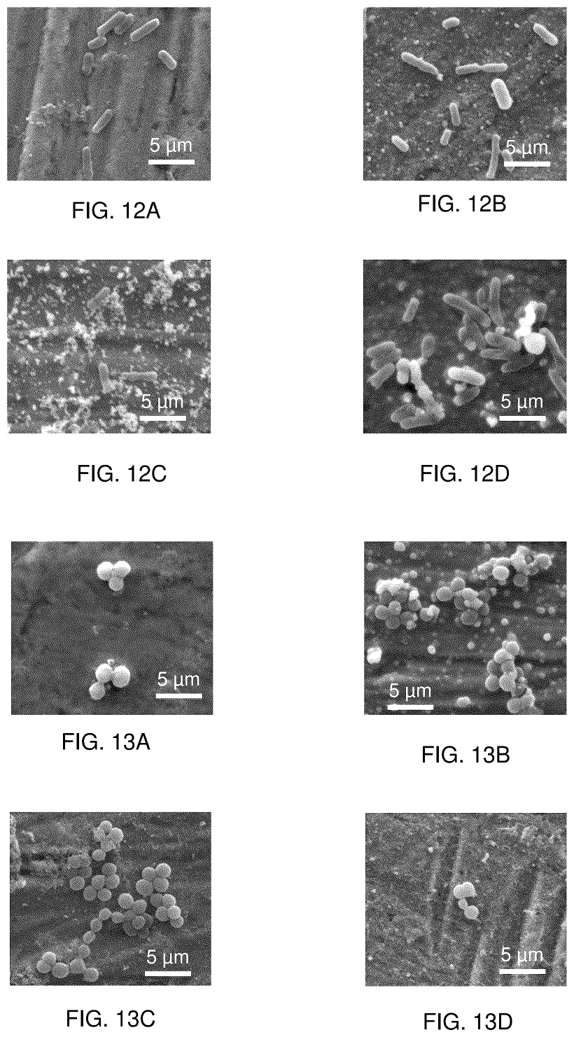

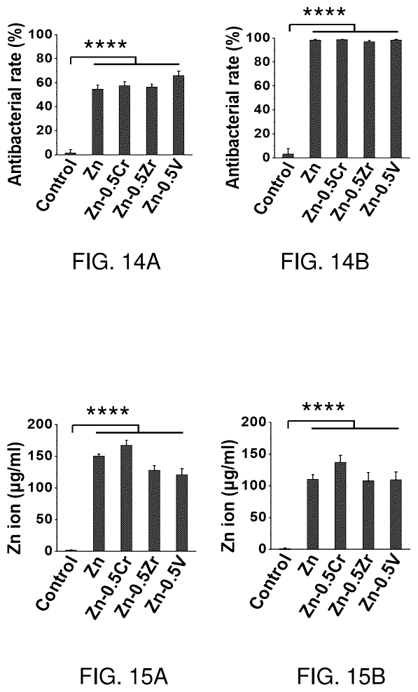

The antibacterial performance of different Zn samples was tested with two bacteria strains E. coli and S. aureus. There was a small amount of bacterial adhesion (see, for example FIGS. 12A-12D (adhesion for E. coli) and FIGS. 13A-13D (adhesion for S. aureus)), and no biofilm formation on the pure Zn and Zn alloy surfaces, indicating their antiadhesion performance for both bacteria strains. There was much less S. aureus adhesion on the pure Zn and Zn-0.5V surfaces when compared with other groups Compared to the control groups, the antibacterial rate significantly increased to more than 50% for E. coli and around 99% for S. aureus when cultured with pure Zn and Zn alloys (see, FIGS. 14A-14B and 15A-15B). The Zn ion concentrations were measured after 24 h of bacterial culture. There were no significant differences in different Zn groups, although the Zn—Cr group showed slightly higher Zn ion release.

### Example 7: Subcutaneous Implantation of Bioresorbable Materials

To compare different tissue responses on pure Zn and its alloys, samples were implanted into the subcutaneous tissues, the aortas, and the femurs in rats. All procedures were approved by the Cornell University IACUC and Stony Brook University IACUC, following NIH guidelines for the care and use of laboratory animals. Before implantation, samples were sterilized by ultraviolet irradiation for 1 hour and subsequently soaked in 70% ethanol for another hour.

### Subcutaneous Implantation

The study of subcutaneous implantation adopted young male CD IGS rats (strain code: 001, 8-10 weeks, body weight=300-325 grams, Charles River Laboratories, Boston, MA). Each group of samples was implanted into three different rats (n=3). Subcutaneous implantation was performed as described, for example, in Y. Jang, Z. Tan, C. Jurey, Z. Xu, Z. Dong, B. Collins, Y. Yun, J. Sankar, Understanding corrosion behavior of Mg—Zn—Ca alloys from subcutaneous mouse model: effect of Zn element concentration and plasma electrolytic oxidation, Mater Sci Eng C Mater Biol Appl 48 (2015) 28-40, and F. Peng, H. Li, D. Wang, P. Tian, Y. Tian, G. Yuan, D. Xu, X. Liu, Enhanced Corrosion Resistance and Biocompatibility of Magnesium Alloy by Mg—Al-Layered Double Hydroxide, ACS Appl Mater Interfaces 8 (51) (2016) 35033-35044. Briefly, five incisions were made to the subcutaneous tissues of the back and subcutaneous pockets by separating subcutaneous layers with muscle layers. Zn and Zn alloy implants were inserted into separated subcutaneous pockets, and the incisions were closed by absorbable sutures.

Zn and its alloys were first implanted into subcutaneous pockets of rats to evaluate the responses of subcutaneous tissues to the alloys. Micro-CT scanning showed similar morphologies and degradation profiles between Zn, Zn-0.5Zr, and Zn-0.5V. However, fractures and increased degradation could be observed in the Zn-0.5Cr group. Accordingly, macroscopic views of the Zn and the other two alloys showed no noticeable difference after three months of subcutaneous implantation, while Zn-0.5Cr broke into two segments. Zn-0.5Cr showed a 5-fold faster degradation rate than the other groups. To evaluate host responses to the implanted alloys, tissues surrounding the alloys were examined with SEM. The SEM images showed a complete cell coverage on the surfaces of all the samples from both axial and cross-section views.

### Example 8: Aortic Implantation of Bioresorbable Materials

Male young CD IGS rats (strain code: 001, 8-10 weeks, body weight=300-325 grams, Charles River Laboratories, Boston, MA) were chosen for the aortic implantation. Each group of samples was implanted into 3 different rats (n=3). Before implantation, samples were sterilized in 70% ethanol for 1 hour for sterilization and then soaked in heparin solution (1 mg/mL in saline) overnight for anticoagulation. Aortic implantation was performed as described in, for example. Fu, Y. Su, Y.-X. Qin, Y. Zheng, Y. Wang, D. Zhu, Evolution of metallic cardiovascular stent materials: A comparative study among stainless steel, magnesium and zinc, Biomaterials 230 (2020) 119641. Briefly, the midline of the abdominal incision was made to expose the abdominal aorta. The aorta was separated from the inferior vena cava. Blood flow in the aorta was blocked with a microvascular clamp. Zn and Zn alloy wire implants were inserted into the aorta with middle parts (6-8 mm in length) within the lumen and two ends (1-2 mm in length) outside of vessel walls. The microvascular clamp was then removed from the aorta to recover the blood flow. The surgical site was closed with absorbable sutures. No anticoagulation or antiplatelet treatments were administrated pre- or post-operatively.

Zn and its alloys were further implanted into the abdominal aortas of rats to investigate vascular responses to the alloys. The overall success rate of the surgeries was 100%, and no surgery-associated complications were observed. The implanted alloys could be easily identified through B mode of ultrasound, which presented as white dots above shadows of ultrasound in the abdominal aortas. In addition, blood flows in the lumens of the aortas existed all the time, and no occlusion occurred over a 3-month observation window, indicating a good hemocompatibility of the implanted alloys. However, the flow rate in the aortas gradually decreased with time in all the groups, with a significant difference observed only between Zn and Zn-0.5Cr groups in Week 2. The degradation rates were 0.05-0.07 mm/y for all the implants, where the Zn-0.5Cr group still showed the highest value. Macroscopic views showed that the two ends of the wires outside of the lumens were wrapped up by fibrotic capsules.

The explanted samples were then analyzed by histology. H&E staining showed that the segments of all alloys in the lumens of the aortas were wrapped by neointimas and the quantification data of the neointimal areas showed no significant difference in different groups. Interfaces between the alloys and the surrounding tissues were clear for Zn, Zn-0.5V, and Zn-0.5Zr. However, the interfaces between Zn-0.5Cr and the surrounding tissues were blurry with numerous necrotic tissues, which indicates a strong inflammation response. For the segments of the alloys outside of the lumens, metal-vascular matrix interfaces were clear for Zn-0.5V but a little blurry for Zn, Zn-0.5Zr, and Zn-0.5Cr due to tissue necrosis. There were larger inflammation areas surrounding the Zn, Zn-0.5Zr, and Zn-0.5Cr wire implants than that of Zn-0.5V implant, with the largest inflammation areas observed in Zn-0.5Cr group. This result suggests that inflammation induced by Zn-0.5V is milder than that caused by Zn, Zn-0.5Zr, or Zn-0.5Cr. Besides, the segments of all the alloys in and outside of the lumens were surrounded by collagens and elastin. Fewer collagens were found in Zn-0.5Cr group than those in Zn-0.5Zr and Zn-0.5V groups, but the elastin contents were similar between different groups. For the segments of the alloys outside of the lumens, the contents of collagen and elastin were comparable between different groups.

Monocytes/macrophages were also investigated by immunofluorescence staining. For segments of the alloys in and outside of lumens, Zn, Zn-0.5Zr, and Zn-0.5V groups had only a few CD68⁺ cells and most of them were close to the alloys. However, there were quite a lot of CD68⁺ cells in the Zn-0.5Cr group, indicating an active inflammation. Samples were further stained with CD206, an M2 macrophage marker. Interestingly, for the segments of all the alloys in and outside of lumens, most cells surrounding the alloys were CD206 positive, with a higher percentage in the Zn-0.5Cr group as compared to the pure Zn group. Since M2 macrophages promote wound healing, monocytes/macrophages may differentiate into M2 macrophages, promoting regeneration of the tissues surrounding the alloys. Finally, SMC and EC coverage on the surface of the alloys were evaluated. For the segments of all the alloys in lumens, most cells surrounding the alloys were αSMA positive, and the cells in direct contact with the blood were eNOS positive despite some background staining. Larger αSMA positive areas could be observed in the Zn-0.5Cr group, while smaller eNOS positive areas were seen in Zn-0.5Cr and Zn-0.5Zr group as compared to pure Zn. On the other hand, for the segments of all the alloys outside of lumens, only SMC layers and endothelium of the abdominal aortas were positive for αSMA and cNOS, respectively.

### Example 9: Femoral Implantation of Bioresorbable Materials

Male young Sprague Dawley rats (8-10 weeks, body weight=300-325 grams, Taconic Biosciences, NY) were used for the in vivo analysis of the femoral implantation, and each group of samples was implanted into 5 different rats (n=5). Briefly, a 2 cm incision was made longitudinally along the lateral side of the right femur. A cylindrical hole (0.3 mm in diameter) was drilled in the femoral condyle perpendicular to the long axis of the femur. Zn and Zn alloy implants were inserted into the cylindrical hole. The incision was closed in layers with sutures.

In vivo degradation and hard tissue compatibility were tested for pure Zn and its alloys following 3-month implantation in femur tissue using micro-CT and cross-sectional SEM imaging together with the elemental compositions. Micro-CT scanning showed similar morphologies and degradation rates (˜0.03 mm/y) between Zn, Zn-0.5Zr, and Zn-0.5V groups, while deep pitting morphology and higher degradation rate (˜0.05 mm/y) were observed in the Zn-0.5Cr group. The SEM images showed the different gray scales, corresponding to the elemental compositions in EDS mapping. Therefore, the new-formed bone tissue and osteoid tissue were identified together with the implants and the degradation products in the SEM images. All implants had similar bone formation and osteoid tissue formation surrounding the Zn implants. Zn—Cr alloy induced a slightly higher bone formation, which might result from its marginally higher degradation rate when compared to other groups. Zn-0.5Zr alloy showed more degradation products in the cross-sectional images, but it did not induce any significant tissue response. All three Zn alloys induced significantly higher bone-implant contact ratios, indicating the higher bone tissue integration. Masson-Goldner and Elastica van Gieson staining were carried out to further study the femur tissue response with pure Zn and its alloys. A brick red color in Masson-Goldner staining and a corresponding light red color in Elastin staining are related to the osteoid tissue. The identification of the tissues through histological staining confirmed the results in SEM images. New-formed bone tissue was also observed within the ostcoid layer, indicating the conversion of osteoid tissue to bone tissue.

While the present disclosure has been particularly shown and described with respect to preferred embodiments thereof, it will be understood by those skilled in the art that the foregoing and other changes in forms and details may be made without departing from the spirit and scope of the present disclosure. It is therefore intended that the present disclosure not be limited to the exact forms and details described and illustrated, but fall within the scope of the appended claims.

What is claimed is:

1. A wound closure device comprising:
a bioresorbable material composed of zinc (Zn) and at least one primary alloying element, wherein the at least one primary alloying element is selected from the group consisting of chromium (Cr), vanadium (V), and zirconium (Zr).

2. The wound closure device of claim 1, wherein the bioresorbable material further comprises at least one secondary alloying element, wherein the at least one secondary alloying element is selected from the group consisting of aluminum (Al), iron (Fe), calcium (Ca), strontium (Sr), silver (Ag), copper (Cu), titanium (Ti), manganese (Mn), selenium (Se), molybdenum (Mo), cobalt (Co), silicon (Si), tin (Sn), nickel (Ni), lithium (Li), sodium (Na), potassium (K), germanium (Ge), rubidium (Rb), tungsten (W), cesium (Ce), scandium (Sc), and yttrium (Y).

3. The wound closure device of claim 2, wherein the bioresorbable material is devoid of magnesium (Mg).

4. The wound closure device of claim 1, wherein the at least one primary alloying element is present in a non-toxic amount.

5. The wound closure device of claim 4, wherein the bioresorbable material comprises from about 0.1 atomic percent to about 12 atomic percent of the least one primary alloying element.

6. The wound closure device of claim 5, wherein the bioresorbable material comprises from about 0.1 atomic percent to about 8 atomic percent of the least one primary alloying element.

7. The wound closure device of claim 1, wherein the bioresorbable material is a binary compound of Zn and Zr.

8. The wound closure device of claim 7, wherein the binary compound comprises from about 0.1 atomic percent to about 12 atomic percent Zr, and the remainder of the binary compound material is Zn.

9. The wound closure device of claim 8, wherein the binary compound comprises about 0.5 atomic percent Zr.

10. The wound closure device of claim 1, wherein the bioresorbable material is a binary compound of Zn and Cr.

11. The wound closure device of claim 10, wherein the binary compound comprises from about 0.1 atomic percent to about 12 atomic percent Cr, and the remainder of the binary compound material is Zn.

12. The wound closure device of claim 11, wherein the binary compound comprises about 0.5 atomic percent Cr.

13. The wound closure device of claim 1, wherein the bioresorbable material is a binary compound of Zn and V.

14. The wound closure device of claim 13, wherein the binary compound comprises from about 0.1 atomic percent to about 12 atomic percent V, and the remainder of the binary compound material is Zn.

15. The wound closure device of claim 14, wherein the binary compound comprises about 0.5 atomic percent V.

16. The wound closure device of claim 1, wherein the at least one primary alloying element is present as an intermetallic phase precipitate in Zn.

17. The wound closure device of claim 1, wherein the at least one primary alloying element forms a local atomic bond with Zn.

18. The wound closure device of claim 1, wherein the bioresorbable material has a compressive yield strength from about 10 MPa to about 1000 MPa.

19. The wound closure device of claim 1, wherein the bioresorbable material has an elastic modulus from about 10 GPa to about 200 GPa.

20. The wound closure device of claim 1, wherein the bioresorbable material has an elongation to failure of from about 1 percent to about 80 percent.

21. The wound closure device of claim 1, wherein the bioresorbable material has a degradation rate of from about 0.01 mm/y to about 1 mm/y.

22. The wound closure device of claim 1, wherein the bioresorbable material is antibacterial.

23. The wound closure device of claim 22, wherein the bioresorbable material exhibits an adhesion for at least one of E. coli and S aureus.

24. The wound closure device of claim 1, wherein the bioresorbable material exhibits an antibacterial rate for E. coli of from about 30 percent to about 100 percent.

25. The wound closure device of claim 1, wherein the bioresorbable material exhibits an antibacterial rate for S. aureus of from about 50 percent to 100 percent.

26. The wound closure device of claim 1, wherein the bioresorbable material is the shape of a staple.

27. The wound closure device of claim 1, wherein the bioresorbable material is implantable into a mammalian body and is configured to close an internal wound.

28. The wound closure device of claim 1, wherein the bioresorbable material is applied on an external surface of a mammalian body and is configured to close an external wound.

29. A method of forming a wound closure device comprising:
mixing, in any order, zinc (Zn) and at least one primary alloying element to provide a blend of Zn and the at least one primary alloying element, wherein the at least one primary alloying element is selected from the group consisting of chromium (Cr), vanadium (V), and zirconium (Zr);
heating the blend to a temperature that melts at least the least one primary alloying element;
cooling the heated blend to provide a bioresorbable material of Zn and the at least one primary alloying element, wherein the bioresorbable material comprises the least one primary alloying element as an intermetallic precipitate in Zn; and
shaping the bioresorbable material into a shape of the wound closure device.

30. The method of claim 29, wherein the mixing further includes adding at least one least one secondary alloying element with the Zn and at least one primary alloying element, wherein the at least one secondary alloying element is selected from the group consisting of aluminum (Al), iron (Fe), calcium (Ca), strontium (Sr), silver (Ag), copper (Cu), titanium (Ti), manganese (Mn), selenium (Se), molybdenum (Mo), cobalt (Co), silicon (Si), tin (Sn), nickel (Ni), lithium (Li), sodium (Na), potassium (K), germanium (Ge), rubidium (Rb), tungsten (W), cesium (Ce), scandium (Sc), and yttrium (Y).

31. The method of claim 29, wherein from about 0.1 atomic percent to about 12 atomic percent of the least one primary alloying element is present in the blend.

32. The method of claim 29, wherein the heating is performed at a temperature from about 100° C. to about 600° C.

33. The method of claim 29, wherein the cooling comprises water quenching at a cooling rate from about 1° C./min to about 100° C./min.

34. The method of claim 29, wherein the shape of the wound closure device is a staple.

35. A method of treating a wounded mammal, the method comprising:
applying a wound closure device to a wound of the mammal, the wound closure device comprising a bioresorbable material composed of zinc (Zn) and at least one primary alloying element, wherein the at least one primary alloying element is selected from the group consisting of chromium (Cr), vanadium (V), and zirconium (Zr); and
closing the wound with the wound closure device.

36. The method of claim 35, wherein the wound is an internal wound, and the applying comprises implanting the wound closure device into a body of the mammal.

37. The method of claim 35, wherein the wound is an external wound, and the applying comprises sewing or stapling the wound closure device into the skin of the mammal.

38. The method of claim 35, wherein the wound closure device is staple, wire or suture.

39. The method of claim 35, wherein the bioresorbable material comprises from about 0.1 atomic percent to about 12 atomic percent of the least one primary alloying element.

40. The method of claim 35, wherein the bioresorbable material further comprises at least one secondary alloying element, wherein the at least one secondary alloying element is selected from the group consisting of aluminum (Al), iron (Fe), calcium (Ca), strontium (Sr), silver (Ag), copper (Cu), titanium (Ti), manganese (Mn), selenium (Se), molybdenum (Mo), cobalt (Co), silicon (Si), tin (Sn), nickel (Ni), lithium (Li), sodium (Na), potassium (K), germanium (Ge), rubidium (Rb), tungsten (W), cesium (Cc), scandium (Sc), and yttrium (Y).
